(19) United States
(12) Patent Application Publication
(10) Pub. No.: US 2025/0075264 A1
(43) Pub. Date: Mar. 6, 2025

(54) DETECTION DEVICE AND NUCLEIC ACID EXTRACTION METHOD

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Beiyuan Fan, Beijing (CN); Ding Ding, Beijing (CN)

(73) Assignees: BEIJING BOE TECHNOLOGY DEVELOPMENT CO., LTD., Beijing (CN); BOE TECHNOLOGY GROUP CO., LTD., Beijing (CN)

(21) Appl. No.: 18/579,541

(22) PCT Filed: Aug. 23, 2022
(86) PCT No.: PCT/CN2022/114199

§ 371 Date: Jan. 16, 2024

(51) Int. Cl. C12Q 1/6844; B01L 3/00

(52) U.S. Cl. CPC C12Q 1/6844; B01L 3/502761; B01L 2200/0668; B01L 2200/16; B01L 2300/0681; B01L 2300/0832; B01L 2300/087; B01L 2400/0478

(57) ABSTRACT

Detection device and nucleic acid extraction method are disclosed. The detection device includes body chamber including first and second surfaces oppositely arranged, cover, piston rotatable plate and piston. The first surface is open. Sub-chambers are provided in circumferential direction of body chamber. End of each sub-chamber away from first surface has first opening. The cover is arranged on the first surface and has second opening connecting first sub-chamber. The piston rotatable plate includes cylindrical structure connected to the second surface, and includes first and second accommodating chambers connected via connecting channel along axial direction of the cylindrical structure. Third surface of the piston rotatable plate facing the body chamber includes at least one third opening connecting the first accommodating chamber. The piston is in slidable connection into the second accommodating chamber, and is synchronously rotatable with the piston rotatable plate in circumferential direction of the second accommodating chamber.

### TECHNICAL FIELD

The present disclosure relates to the technical field of reagent detection, and in particular, to a detection device and a nucleic acid extraction method.

### BACKGROUND

A kit is a box for containing a chemical reagent such as chemical component, drug residue, virus species, and the like. The kit in the related art has a complicated structure, and an independent driving structure is used for moving a corresponding component, which results in a high cost.

### SUMMARY

To address the technical issue, the present disclosure provides a detection device and a nucleic acid extraction method, which address the technical issue that the kit structure is complex and the cost thereof is high.

To achieve this, embodiments of the present disclosure adopt the following technical solutions. A detection device includes a body chamber, a cover, a piston rotatable plate, and a piston;

The body chamber includes a first surface and a second surface which are opposite to each other, and the first surface is an open surface; along a circumferential direction of the body chamber, the body chamber includes a plurality of sub-chambers, and an end of each of the plurality of sub-chambers that is away from the first surface is provided with a first opening.

The cover is provided to cover the first surface of the body chamber, and a second opening is arranged on the cover, the second opening is configured to connect with a first sub-chamber of the plurality of sub-chambers.

The piston rotatable plate includes a cylindrical structure connected to the second surface of the body chamber; in an axial direction of the cylindrical structure, the piston rotatable plate includes a first accommodating chamber and a second accommodating chamber, the first accommodating chamber and the second accommodating chamber are connected with each other via a connecting channel, and a third surface of the piston rotatable plate facing to the body chamber includes at least one third opening in connection with the first accommodating chamber.

The piston is in slidable connection into the second accommodating chamber in an axial direction of the second accommodating chamber, and the piston is synchronously rotatable with the piston rotatable plate in a circumferential direction of the second accommodating chamber.

Optionally, in the circumferential direction of the body chamber, the second surface of the body chamber is provided with at least one concentric annular groove recessed toward an interior of the body chamber, each annular groove is provided with at least one first opening, and each third opening is in slidable connection into a corresponding one annular groove.

Optionally, the at least one annular groove includes a first annular groove and a second annular groove, the first opening includes a first sub-opening located at the first annular groove and at least one second sub-opening located at the second annular groove, the at least one second sub-opening is arranged to be spaced apart on the second annular groove.

Optionally, an orthographic projection of the first sub-opening onto the second annular groove is located between two adjacent second sub-openings.

Optionally, the at least one third opening includes a fourth sub-opening and a fifth sub-opening, the fourth sub-opening is in slidable connection into the first annular groove and the fifth sub-opening is in slidable connection into the second annular groove, a first pipeline is arranged between the fourth sub-opening and the first accommodating chamber, and a second pipeline is arranged between the fifth sub-opening and the first accommodating chamber.

Optionally, a first filtering membrane is arranged in the first pipeline, the first filtering membrane is fixed on an inner wall of the first pipeline, and an orthographic projection of the first filtering membrane in an axial direction of the first pipeline fully covers the first pipeline.

Optionally, a second filtering membrane is arranged in the connecting channel between the first accommodating chamber and the second accommodating chamber, the second filtering membrane is fixed on an inner wall of the connecting channel, and an orthographic projection of the second filtering membrane in an axial direction of the connecting channel fully covers the connecting channel.

Optionally, the at least one annular groove further includes a third annular groove, the at least one third opening further includes a sixth sub-opening and a seventh sub-opening in slidable connection into the third annular groove, the sixth sub-opening and the seventh sub-opening are arranged to be spaced apart from each other, and a channel is arranged between the sixth sub-opening and the seventh sub-opening.

Optionally, the plurality of sub-chambers include a second sub-chamber, a side wall of the second sub-chamber is provided with a first outlet and a first inlet, a first opening located in the second sub-chamber includes a second inlet and a second outlet, a pipeline for connecting the first inlet and the second outlet and a pipeline for connecting the second inlet and the first outlet are arranged in the second sub-chamber, and the second inlet and the second outlet are arranged on the third annular groove.

Optionally, a clamping slot is arranged at a center of the third surface, and a connecting column that fits with the clamping slot is arranged at a center of the second surface.

Optionally, the connecting column extends to the first surface, an end of the connecting column away from the second surface is arranged to be flush with the first surface, and the plurality of sub-chambers are arranged around the connecting column; and the connecting column is a hollow structure, and the cover is provided with a fourth opening in connection with a center of the connecting column.

Optionally, the first opening includes at least two connecting pieces connected against each other, which are formed by cutting at a first position of the annular groove, the third opening is made of an elastic material, and a length of the third opening in a direction perpendicular to the third surface is greater than or equal to a depth of the annular groove.

Optionally, the first position of the annular groove is recessed towards the interior of the body chamber to be a first groove, the first opening includes at least two connecting pieces connected against each other, which are formed by cutting at a bottom portion of the first groove, the third opening is made of an elastic material, the length of the third opening in the direction perpendicular to the third surface is greater than the depth of the annular groove, and the length of the third opening in the direction perpendicular to the third surface is greater than or equal to a depth of the first groove.

Optionally, a plurality of first projections are arranged at intervals on at least one inner wall of the sub-chamber.

Optionally, at least one pillar is arranged at a bottom wall of the sub-chamber located at the second surface, and a plurality of second projections are provided at intervals on a peripheral surface of the at least one pillar.

Optionally, a plurality of third projections are arranged at intervals on an inner wall of the first accommodating chamber.

Optionally, the piston includes a movable portion and a transmission portion connected to the movable portion, the movable portion extends into the second accommodating chamber, an orthographic projection of the movable portion in the axial direction of the second accommodating chamber fully covers the second accommodating chamber, the transmission portion is located outside the second accommodating chamber, and the transmission portion includes a transmission gear.

Optionally, a sliding groove is arranged on the inner wall of the second accommodating chamber along the axial direction of the second accommodating chamber, and a sliding block that fits the sliding groove is arranged on a peripheral surface of the movable portion.

Optionally, the movable portion is made of an elastic material, and a cross-sectional shape of the movable portion is consistent with a shape of the second accommodating chamber in a direction parallel to the first surface.

An embodiment of the present disclosure further provides a nucleic acid extraction method, in which a detection is performed by using the detection device as mentioned above, a magnet structure is contained in a clamping slot of the piston rotatable plate, the first sub-chamber is a sample chamber for containing a magnetic bead buffer solution, and the plurality of sub-chambers further include: a first reagent chamber for containing a binding solution, a second reagent chamber for containing a cleaning solution, a third reagent chamber for containing an eluent, a waste liquid chamber, a PCR reagent chamber for containing a PCR reagent, and a detection chamber for containing a detection chip.

The method includes the following steps:
placing a sample into the sample chamber, to cause the sample to be mixed with the magnetic bead buffer solution to obtain a first magnetic bead mixed solution;
rotating the piston thereby driving the piston rotatable plate to rotate synchronously, to cause the third opening of the piston rotatable plate to be opposite to a first opening of the sample chamber; moving the piston in a direction away from the first accommodating chamber, to cause the magnetic bead mixed solution to enter the first accommodating chamber; and adsorbing magnetic beads by the magnet structure;
rotating the piston rotatable plate, to cause the third opening to be opposite to a first opening of the waste liquid chamber; and moving the piston in a direction towards the first accommodating chamber, to cause waste liquid from which the magnetic beads is separated to enter the waste liquid chamber;
rotating the piston rotatable plate, to cause the third opening to be opposite to a first opening of the first reagent chamber; moving the piston in the direction away from the first accommodating chamber, to cause the binding solution in the first reagent chamber to enter the first accommodating chamber; and releasing the adsorbed magnetic beads by the magnet structure, so that the magnetic beads are mixed with the binding solution to obtain a second mixed solution;
rotating the piston rotatable plate, to cause the third opening to be opposite to the first opening of the waste liquid chamber; adsorbing the magnetic beads by the magnet structure; and moving the piston in the direction towards the first accommodating chamber, to cause waste liquid from which the magnetic beads is separated to enter the waste liquid chamber;
rotating the piston rotatable plate, to cause the third opening to be opposite to a first opening of the second reagent chamber; moving the piston in the direction away from the first accommodating chamber, to cause the cleaning solution in the second reagent chamber to enter the first accommodating chamber; and releasing the adsorbed magnetic beads by the magnet structure, so that the magnetic beads are mixed with the cleaning solution to obtain a third mixed solution;
rotating the piston rotatable plate, to cause the third opening to be opposite to the first opening of the waste liquid chamber; adsorbing the magnetic beads by the magnet structure; moving the piston in the direction towards the first accommodating chamber, to cause waste liquid from which the magnetic beads is separated to enter the waste liquid chamber;
rotating the piston rotatable plate, to cause the third opening to be opposite to a first opening of the third reagent chamber; moving the piston in the direction away from the first accommodating chamber, to cause the eluent in the third reagent chamber to enter the first accommodating chamber; and releasing the adsorbed magnetic beads by the magnet structure, so that the magnetic beads is mixed with the eluent and nucleic acid are separated from the magnetic beads to obtain a nucleic acid mixed solution;
rotating the piston rotatable plate, to cause the third opening to be opposite to a first opening of the PCR reagent chamber; moving the piston in the direction towards the first accommodating chamber, to cause the nucleic acid mixed solution to enter the PCR reagent chamber, wherein the nucleic acid mixed solution mixes with the PCR reagent in the PCR reagent chamber to obtain a PCR reagent mixed solution;
moving the piston in the direction away from the first accommodating chamber, to cause the PCR reagent mixed solution to enter the first accommodating chamber; and
rotating the piston rotatable plate and moving the piston is the direction towards the first accommodating chamber, to cause the PCR reagent mixed solution to enter a detection region for detection.

Various advantageous technical effects may be achieved with the solution of the present disclosure. For example, the piston can perform stretch-out and draw-back movement in the second accommodating chamber, and the piston can also rotate to drive the rotation of the piston rotatable plate, in this way, the switching to different reagent chambers can be realized. Namely, the detection on the corresponding sample can be realized by only controlling the piston to perform corresponding movements, thereby simplifying the structure and reducing the cost of the detection device.

### BRIEF DESCRIPTION OF THE DRAWINGS

FIG. 1 shows an exploded structural view of a kit in an embodiment of the present disclosure;

FIG. 2 shows a schematic top view of a body chamber in an embodiment of the present disclosure;

FIG. 3 shows a schematic diagram of a bottom of the body chamber in an embodiment of the present disclosure;

FIG. 4 shows a schematic diagram of a portion of a structure of the body chamber in an embodiment of the present disclosure;

FIG. 5 shows a schematic diagram of a structure of a piston rotatable plate in an embodiment of the present disclosure;

FIG. 6 shows a schematic diagram of an internal structure of the body chamber in an embodiment of the present disclosure;

FIG. 7 is a schematic diagram showing a connection state for the piston rotatable plate and the body chamber;

FIG. 8 shows a schematic diagram showing a first opening being opposite a third opening in an embodiment of the present disclosure;

FIG. 9 is a schematic structural diagram of a cover according to an embodiment of the present disclosure;

FIG. 10 is a schematic diagram of a first opening in an embodiment of the present disclosure; and

FIG. 11 is a schematic diagram showing a corresponding connection state for the piston rotatable plate and the body chamber.

### DETAILED DESCRIPTION

To make the objective, technical solution and advantages of embodiments of the present disclosure more apparent, a detailed description for the embodiments of the present disclosure will be given clearly in conjunction with the appended drawings. It is to be understood that the described embodiments are part, but not all, of the embodiments of this disclosure. Based on the described embodiments of the present disclosure, all other embodiments that can be obtained by one of ordinary skill in the art fall within the protection scope of the present disclosure.

In describing the present disclosure, It is to be noted that the terms “central”, “upper”, “lower”, “left”, “right”, “vertical”, “horizontal”, “inner”, “outer”, and the like indicate orientations or positional relationships based on the orientations or positional relationships shown in the figure, these terms are used merely for the purpose of facilitating and simplifying description of the present disclosure, and do not indicate or imply that the referenced devices or elements must have a particular orientation, be constructed and operated in a particular orientation, and thus should not be construed as limitation on the present disclosure. Furthermore, the terms “first”, “second”, and “third” are used for descriptive purposes only and are not to be construed as indicating or implying relative importance.

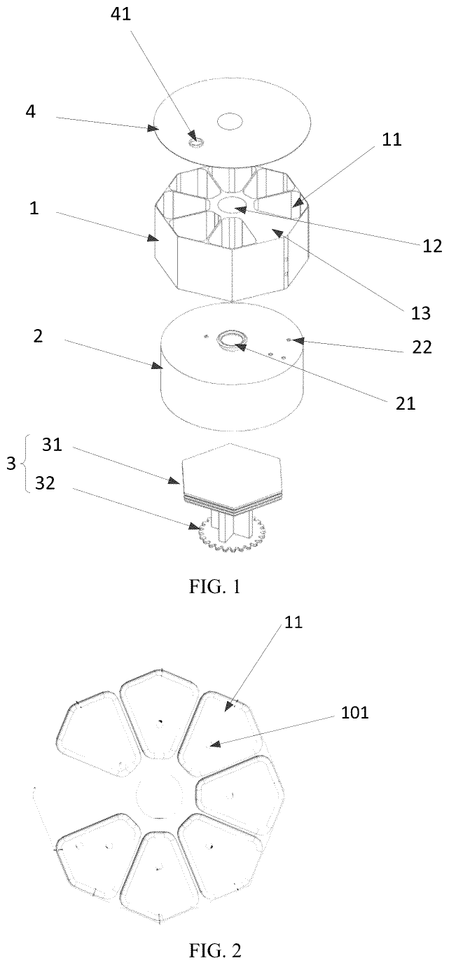

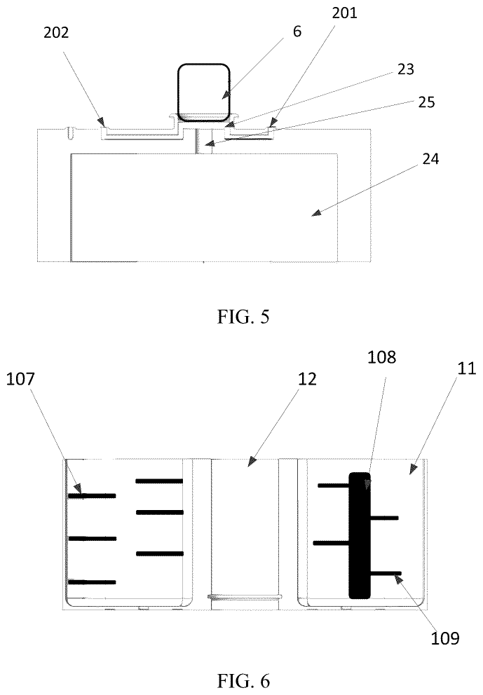

As shown in FIGS. 1 to 11, an embodiment provides a detection device, including a body chamber 1, a cover 4 and a piston rotatable plate 2.

The body chamber 1 includes a first surface and a second surface which are opposite to each other, and the first surface is an open surface. Along a circumferential direction of the body chamber 1, the body chamber 1 includes a plurality of sub-chambers 11. An end of each of the sub-chambers 11 that is away from the first surface is provided with a first opening 101. The specific functional arrangement of the plurality of the sub-chambers 11 can be arranged according to actual needs, for example, a sample chamber and at least one reagent chamber containing a preset reagent may be included in the sub-chambers.

The cover 4 covers the first surface of the body chamber 1, where the cover 4 is provided with a second opening 41 to connect with a first sub-chamber of the plurality of sub-chambers, and the first sub-chamber may be a sample chamber which may facilitate the insertion of a sample, but the present disclosure is not limited thereto.

The piston rotatable plate 2 includes a cylindrical structure connected to the second surface of the body chamber 1. In an axial direction of the cylindrical structure, the piston rotatable plate 2 including a first accommodating chamber 23 and a second accommodating chamber 24, the first accommodating chamber 23 and the second accommodating chamber 24 connect with each other via a connecting channel 25, a third surface of the piston rotatable plate 2 facing the body chamber 1 includes at least one third opening 22 in connection with the first accommodating chamber 23.

A piston 3 is in slidable connection into the second accommodating chamber 24 in an axial direction of the second accommodating chamber 24, and the piston 3 is synchronously rotatable with the piston rotatable plate 2 in a circumferential direction of the second accommodating chamber 24.

When performing a detection, the detection device is to be placed into a corresponding detection instrument to perform a corresponding operation. In this embodiment, the piston 3 may control the piston rotatable plate 2 to rotate, to make the third opening 22 being opposite to the first opening 101, such that the first accommodating chamber 23 is in connection with a corresponding sub-chamber 11; and the piston 3 performs a stretch-out and draw-back movement in the second accommodating chamber 24, so that a medium (the medium can be a sample, a mixture of a sample and a corresponding reagent, a mixed solution after a reaction, etc.) in the corresponding sub-chamber 11 enters the first accommodating chamber 23, alternatively, a medium in the first accommodating chamber 23 enters the corresponding sub-chamber 11, so as to perform a corresponding processing such as purifying, or detecting the sample with different reagents.

There is no need to add a separate driving structure for the rotation of the piston rotatable plate 2, because the driving structure for controlling the stretch-out and draw-back movement of the piston 3 and the driving structure for controlling the rotation of the piston rotatable plate 2 are integrated in one structure, so as to realize connections between the first accommodating chamber 23 and different sub-chambers 11, such that reactions and exchanges between the reagents accommodated in the different sub-chambers 11 can be done.

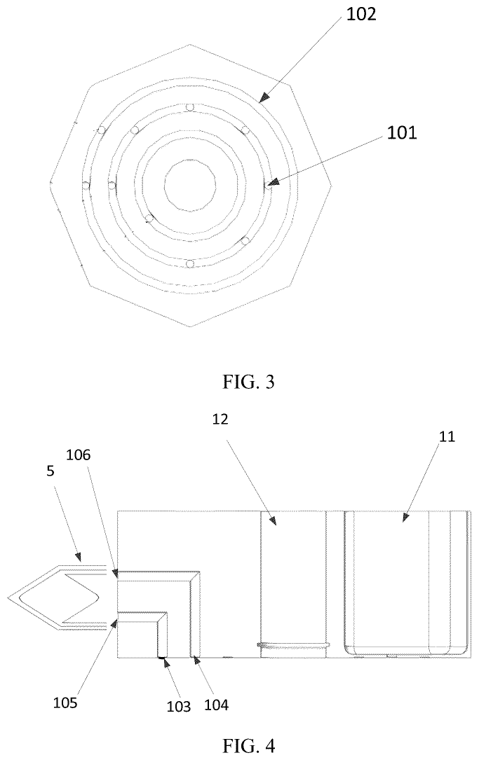

Referring to FIG. 3, in an exemplary embodiment, in the circumferential direction of the body chamber 1, the second surface of the body chamber 1 is provided with at least one concentric annular groove 102 recessed toward an interior of the body chamber 1, each annular groove 102 is provided with at least one first opening 101, and each third opening 22 is in slidable connection into one of the annular grooves 102.

The third opening 22 protrude from the third surface, and the third opening 22 extends into a corresponding annular groove 102 and can rotate along the corresponding annular groove 102.

The provision of the annular groove 102 ensures a rotational trajectory of the piston rotatable plate 2, which can prevent an offset of the piston rotatable plate 2, and ensure the first opening 101 to be opposite to a corresponding third opening 22.

In an exemplary embodiment, the at least one annular groove 102 includes a first annular groove and a second annular groove, and the first opening 101 includes a first sub-opening in the first annular groove and at least one second sub-opening in the second annular groove, the second sub-openings are spaced apart in the second annular groove.

When a plurality of third openings 22 are provided, to prevent multiple third openings 22 from being in connection with corresponding sub-chambers, in this embodiment, only one third opening 22 is in connection with a corresponding sub-chamber when the piston rotatable plate 2 is rotated once i.e., only one sub-chamber is in connection with the first accommodating chamber 23, but the present disclosure is not limited thereto.

In an exemplary embodiment, an orthographic projection of the first sub-opening onto the second annular groove is located between two adjacent the second sub-openings.

In an exemplary embodiment, the at least one third opening includes a fourth sub-opening and a fifth sub-opening, the fourth sub-opening is in slidable connection into the first annular groove and the fifth sub-opening is in slidable connection into the second annular groove, a first pipeline is arranged between the fourth sub-opening and the first accommodating chamber 23, and a second pipeline is arranged between the fifth sub-opening and the first accommodating chamber 23.

In an embodiment, the plurality of sub-chambers 11 include a waste liquid chamber, the third opening 22 includes a fourth sub-opening 201 and a fifth sub-opening 202 in connection with the waste liquid chamber. The first opening 101 (namely, the first sub-opening) in connection with the waste liquid chamber is arranged on the first annular groove. The plurality of sub-chambers include a reagent chamber containing a preset reagent, a second sub-opening in connection with the reagent chamber is arranged on the second annular groove, and the fourth sub-opening 201 is movably coupled to the first annular groove. The fifth sub-opening 202 is movably coupled to the second annular groove. When the fourth sub-opening 201 is connected to the first sub-opening, the fifth sub-opening 202 is not connected with a corresponding second sub-opening, and waste liquid in the first accommodating chamber 23 may only enter the waste liquid chamber.

It is to be noted that the waste liquid chamber is provided, so that after the sample is subjected to a mixture and reaction with one type of reagent, the required medium and impurities for separation can be separated from each other, so as not to affect the accuracy of processing in the next step.

In an exemplary embodiment, a first filtering membrane is arranged in the first pipeline, the first filtering membrane is fixed to an inner wall of the first pipeline, and an orthographic projection of the first filtering membrane in an axial direction of the first pipeline fully covers the first pipeline.

In an embodiment, the plurality of sub-chambers includes a sample chamber, where the sample chamber contains a magnetic bead buffer solution, and the magnetic bead buffer solution is to be mixed with the sample to obtain magnetic beads combined with the substance to be detected in the sample. A side of the first accommodating chamber 23 away from the second accommodating chamber 24 is provided with a clamping slot 21, and the clamping slot 21 contains a magnet structure 6 for adsorbing the magnetic beads such that the magnetic beads are separated from the liquid.

The first filtering membrane is arranged in the first pipeline, and the hole size of the first filtering membrane is smaller than the size of the magnetic beads. By arranging the first filtering membrane, the magnetic beads combined with the substance to be detected can be prevented from entering the waste liquid chamber through the first pipeline which may affect the detection accuracy.

It is noted that the hole size of the first filtering membrane is slightly smaller than the diameter of the magnetic bead, and but is larger than the diameter of biological macromolecules (such as nucleic acid and protein). After the sample is added and the piston performs multiple times of piston motions, the nucleic acid in the sample will be adsorbed by the magnetic beads and thus remain in the first accommodating chamber. When cleaning the magnetic beads with different liquids, a large amount of liquid will pass through the first accommodating chamber to wash the magnetic beads. Afterwards, when performing an elution, multiple times of piston motions is to be performed to ensure a full elution for the magnetic beads.

It should be noted that in this embodiment, extracting the substance to be detected from the sample is achieved based on the magnetic beads, which is applicable to the sample in less amount (there is a smaller amount demand for the sample, i.e., a high concentration of nucleic acid can be extracted from a minor amount of material), and the processing requirements are lower, which is convenient for industrialization.

In an exemplary embodiment, a second filtering membrane is arranged in a connecting channel between the first accommodating chamber 23 and the second accommodating chamber 24, the second filtering membrane is fixed to an inner wall of the connecting channel, and an orthographic projection of the second filtering membrane in the axial direction of the connecting channel fully covers the connecting channel.

The hole size of the second filtering membrane is smaller than the diameter of the magnetic bead. After the sample is mixed with the magnetic bead buffer solution to obtain a first mixed solution, under a cooperation of the rotation of the piston rotatable plate 2 and the stretch-out and draw-back movement (piston motion) of the piston 3, the sample chamber is opposite to the third opening 22, so that the first mixed solution enters the first accommodating chamber 23, then the magnetic beads are adsorbed by the magnet structure 6, and the waste liquid separated from the magnetic beads enters the second accommodating chamber 24. Afterwards, the piston rotatable plate 2 rotates, so that the first opening 101 (namely, the first sub-opening) of the waste liquid chamber is opposite to the corresponding third opening 22 (namely, the fourth sub-opening). The piston 3 moves in a direction towards the first accommodating chamber 23, so that the waste liquid enters the waste liquid chamber, and the second filtering membrane is provided to prevent the magnetic beads from entering the waste liquid chamber.

It should be noted that the magnet structure 6 may be an electromagnet, which may be controlled to generate or not generate a magnetic force under the control of a switch, so as to switch between a state for magnetic bead adhesion and a state for magnetic bead deadhesion.

In an embodiment, the plurality of sub-chambers 11 include a waste liquid chamber, the first pipeline is provided with a first filtering membrane, the hole size of the first filtering membrane is smaller than the diameter of the magnetic bead. A connecting channel 25 between the first accommodating chamber 23 and the second accommodating chamber 24 is provided with a second filtering membrane, the hole size of the second filtering membrane is smaller than the diameter of the magnetic bead.

In this embodiment, the magnetic beads are located on the side of the first accommodating chamber 23 away from the second accommodating chamber 24 by the adsorption action of the magnet structure 6, and the first filtering membrane and the second filtering membrane cooperate to further effectively block the magnetic beads from entering the waste liquid chamber.

In an exemplary embodiment, the at least one annular groove further includes a third annular groove, and the at least one third opening further includes a sixth sub-opening and a seventh sub-opening which are in slidable connection into the third annular groove, the sixth sub-opening is spaced apart from the seventh sub-opening, and a channel is arranged between the sixth sub-opening and the seventh sub-opening.

In an exemplary embodiment, the plurality of the sub-chambers includes a second sub-chamber, a side wall of the second sub-chamber is provided with a first outlet 105 and a first inlet 106, first openings in the second sub-chamber includes a second inlet 103 and a second outlet 104, the second sub-chamber is provided with a pipeline for connecting the first inlet 106 and the second outlet 104, and a pipeline for connecting the second inlet 103 and the first outlet 105, the second inlet 103 and the second outlet 104 are arranged on the third annular groove.

As the piston rotatable plate rotates, the second sub-chamber is connected to the first outlet 105 through the sixth sub-opening, the seventh sub-opening 203, and the second inlet 103. The first inlet 106 and the second outlet 104 is connected to the first accommodating chamber 23 the third opening 22 to form a loop.

In an embodiment, the body chamber 1 includes a detection region 13 and the plurality of sub-chambers 11 distributed along the circumferential direction of the body chamber 1, the second sub-chamber may be the detection region 13, and the plurality of sub-chambers 11 include a PCR reagent chamber, where the PCR reagent chamber contains a PCR reagent for mixing with a product subjected to treatments with various preset reagents.

Referring to FIG. 11, after the sample is treated with reagents such as a magnetic bead buffer solution, a binding solution, a cleaning solution and an eluent to obtain a nucleic acid product, the piston rotatable plate 2 is rotated so that the nucleic acid product enters the PCR reagent chamber and is mixed with the PCR reagent to obtain a PCR reagent mixed solution. By rotating the piston rotatable plate 2 again, the PCR reagent chamber is in connection with the first outlet 105 through the sixth sub-opening, the seventh sub-opening 203 and the second inlet 103, and the first inlet 106 and second outlet 104 are in connection with the first accommodating chamber 23 through the third opening 22 to form a loop. Under the cooperation of the stretch-out and draw-back movement of the piston 3, the PCR reagent mixed solution in the PCR reagent chamber enters a detection chip from the PCR reagent chamber through the sixth sub-opening, the seventh sub-opening, the second inlet 103 and the first outlet 105. To ensure that a housing region for containing the detection chip is filled, there may be an overflowing PCR reagent mixed solution, and the overflowing nucleic acid product enters the first accommodating chamber 23 through the first inlet 106, the second outlet 104 and a corresponding third opening 22 on the piston rotatable plate. Afterwards, by rotating the piston rotatable plate again, the first opening of the waste liquid chamber is in connection with the first accommodating chamber 23, and under the cooperation of the stretch-out and draw-back movement of the piston, a residual liquid for the PCR reagent mixed solution in the first accommodating chamber enters the waste liquid chamber.

In an exemplary embodiment, the plurality of sub-chambers includes a product chamber for receiving a product treated with various reagents, and the cover 4 includes a product outlet corresponding to the product chamber for facilitating removal of the product.

Illustratively, the PCR reagent chamber and the product chamber may be integrated, i.e. the PCR reagent chamber is reused as the product chamber. The nucleic acid product, which is obtained after the sample is treated with reagents such as the magnetic bead buffer solution, the binding solution, the cleaning solution and the eluent, can directly enter the PCR reagent chamber, thereby reducing an additional product chamber and simplifying the structure.

It should be noted that the detection region may be integrated in the body chamber, or provided outside the body chamber. In other words, the detection region 13 for containing the detection chip 5 may be provided in a detection instrument using the detection device. In the embodiment where the detection region for containing the detection chip is provided in the detection instrument using the detection device, the body chamber 1 includes the detection region 13, and a reagent chamber distribution region provided with the plurality of sub-chambers 11. A first outlet 105 and a first inlet 106 are arranged on the side wall of the body chamber 1. The third surface of the piston rotatable plate is provided with a sixth sub-opening and a seventh sub-opening, and the sixth sub-opening and the seventh sub-opening are in connection through a pipeline arranged in the piston rotatable plate.

It should be noted that in an embodiment, the third surface is provided with a fourth sub-opening 201 which may be opposite to a waste liquid chamber, a fifth sub-opening 202 which may be opposite to a reagent chamber (for the PCR reagent chamber, both the fifth sub-opening and the sixth sub-opening are provided), and a seventh sub-opening (in connection with the sixth sub-opening) which may be opposite to the first opening 101 of the detection region 13, so as to avoid the mixture of reagents.

In the exemplary embodiment, the center of the third surface is provided with a clamping slot 21, and the center of the second surface is provided with a connecting column 12 which fits the clamping slot 21.

In an exemplary embodiment, the connecting column extends towards the first surface, an end of the connecting column 12 away from the second surface is arranged to be flush with the first surface, and the plurality of sub-chambers are arranged around the connecting column 12.

The connecting column 12 has a hollow structure, and the cover 4 is provided with a fourth opening 42 in connection with a center of the connecting column 12.

In one embodiment, the plurality of sub-chambers include a sample chamber, where a magnetic bead mixed solution is contained in the sample chamber, a magnet structure 6 is contained in the clamping slot 21, and the magnet structure 6 can be put in or taken out through the fourth opening 42.

In an exemplary embodiment, the clamping slot 21 protrudes from the third surface, a clamping receiving slot is provide at a center of the second surface of the body chamber 1 facing the piston rotatable plate 2, the clamping receiving slot is connected to a central through hole of the connecting column 12, and an area of a bottom of the clamping receiving slot is greater than a radial cross-sectional area of the connecting column 12, so that the clamping slot 21 can extend into the clamping receiving slot and connect with the clamping receiving slot.

An opening is provided at the center of the second surface of the body chamber 1, the clamping receiving slot is formed at a position extending from the opening away from the second surface, an area of a cross section of the clamping receiving slot is greater than an area of a radial cross section of the connecting column 12 in a direction parallel to the second surface, and the clamping slot 21 may be fastened to the bottom of the clamping receiving slot.

In one embodiment, an annular sliding rail is provided at the bottom of the clamping receiving slot, and the clamping slot 21 is rotatably arranged in the annular sliding rail.

In one embodiment, an axial center of the connecting column 12 coincides with an axial center of the clamping slot 21, which may be favorable for putting in or taking out the magnet structure 6.

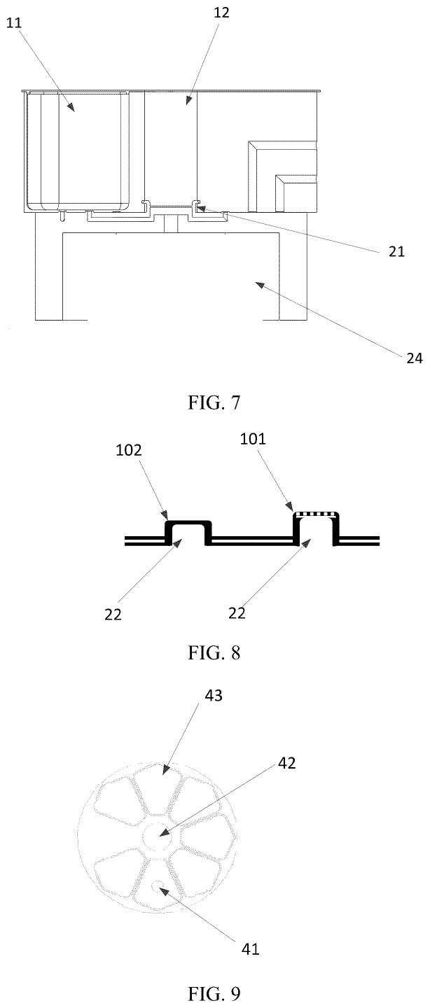

Referring to FIG. 7, in an embodiment, in the direction parallel to the second surface, the area of the cross section of the clamping receiving slot is smaller than the area of the radial cross section of the connecting column 12, an annular clamping receiving sub-slot is provided around the clamping receiving slot, and an inner side wall of the clamping slot 21 is fastened to the annular clamping receiving sub-slot.

In an exemplary embodiment, the first opening 101 includes at least two connecting pieces connected against each other, which are formed by performing a cutting process at a first position of the annular groove 102, the third opening 22 is made of an elastic material, and a length of the third opening 22 in a direction perpendicular to the third surface is greater than or equal to a depth of the annular groove 102.

The first opening 101 is formed by at least two connecting pieces connected against each other, and when being opposite to the third opening 22, the force applied by the third opening 22 on the first opening 101 is zero, and when the piston 3 performs a stretch-out and draw-back movement, a force in a direction away from the piston rotatable plate 2 or a force in a direction facing the piston rotatable plate 2 is applied on the first opening 101, to make the first opening 101 open.

With reference to FIG. 8, two third openings 22, two annular grooves 102, and a first opening 101 located on one of the annular grooves 102 are illustrated in FIG. 8, where the length of the third opening 22 in the direction perpendicular to the third surface is greater than the depth of the annular groove 102, and when one of the third openings 22 (the third opening 22 on the right side) is opposite to the first opening 101, the third opening 22 applies a force on the first opening 101 in a direction away from the piston rotatable plate 2, pushing the first opening 101 to be open. The third opening 22 on the left side is compressed in the corresponding annular groove 102, and since the third opening 22 is made of an elastic material such as rubber, even if the length of the third opening 22 in the direction perpendicular to the third surface is greater than the depth of the annular groove 102, the movement of the third opening 22 in the annular groove 102 will not be affected.

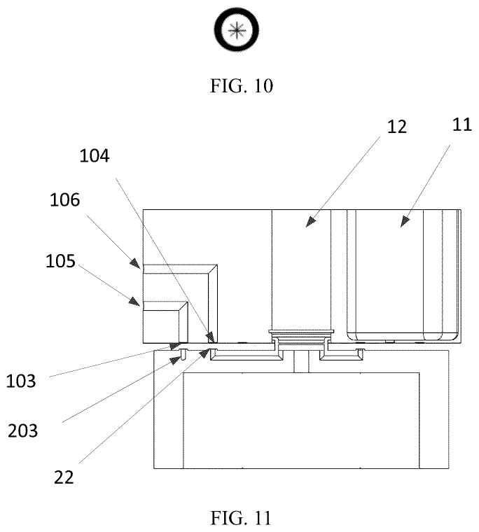

Referring to FIG. 10, in one embodiment, the first opening 101 has a plurality of slit openings extending from center to edge. The first opening 101 is made of an elastic material. In a state that no force is applied, the first opening 101 is in a close state due to the elastic force, and the reagent contained in the sub-chamber is not exposed. In a state that the force is applied, the first opening 101 is opened to connect with the third opening 22.

Note that the material of the first opening 101 may be rubber, silica gel, butyronitrile, etc. but the present disclosure is not limited thereto, as long as the gravity of the reagent in the reagent chamber cannot make the first opening 101 open.

It should be noted that a difference between the length of the third opening 22 in the direction perpendicular to the third surface and the depth of the annular groove 102 is less than a preset value, so as to avoid affecting the sliding of the third opening 22 when the third opening 22 is opposed to the first opening 101.

The preset value may be set according to actual needs, and may be, for example, 0.1 mm to 0.2 mm, but the present disclosure is not limited thereto.

In an embodiment in which the length of the third opening 22 in the direction perpendicular to the third surface is equal to the depth of the annular groove 102, the first opening 101 cannot be opened when the third opening 22 is opposed to the first opening 101, and in this case, a force may be applied to the first opening 101 to open the first opening 101 by the stretch-out and draw-back movement of the piston 3.

In an exemplary embodiment, a first position of the annular groove 102 is recessed toward the interior of the body chamber 1 to form a first groove, the first opening 101 includes at least two connecting pieces connected against each other, which are formed by a cutting process at a bottom of the first groove. the third opening is made of an elastic material, a length of the third opening in the direction perpendicular to the third surface is greater than a depth of the annular groove, and the length of the third opening in the direction perpendicular to the third surface is greater than or equal to a depth of the first groove.

The length of the third opening 22 in the direction perpendicular to the third surface is greater than the depth of the first groove, when the third opening 22 is opposite to the first opening 101, the third opening 22 applies a force to the first opening 101 in a direction away from the piston rotatable plate 2, to push the first opening 101 to be open. Since the third opening 22 is made of an elastic material such as rubber, even if the length of the third opening 22 in the direction perpendicular to the third surface is greater than the depth of the first groove, the movement of the third opening 22 in the annular groove 102 will not be affected.

It should be noted that a difference between the depth of the first groove and the depth of the remaining annular grooves is less than a preset value which for example may be 0.1 mm. A difference between the length of the third opening 22 in the direction perpendicular to the third surface and the depth of the first groove is also less than a preset value which for example may be 0.1 mm. In this way, the difference between the depth of the first groove and the depth of the remaining annular grooves, or the difference between the length of the third opening 22 in the direction perpendicular to the third surface and the depth of the first groove cannot be too large from affecting a rotation of the third opening 22 along the annular groove 102.

Referring to FIG. 6, in an exemplary embodiment, multiple first projections 107 are arranged at intervals on at least one inner wall of the sub-chamber 11.

After the sample is mixed with the magnetic bead buffer solution and enters the first accommodating chamber 23, the separated waste liquid enters the waste liquid chamber. The reagent in one reagent chamber may enter the first accommodating chamber 23 and mix with the magnetic beads twice, this process can enhance the mixing effect of the magnetic beads and the reagent. The shape of the first projection 107 may be a dot shape, a column shape, or a spiral shape, which is not limited herein; and the arrangement of the first projections 107 can enhance the mixing effect of various reagents.

Illustratively, the first projection 107 extends in a direction perpendicular to an axial direction of the body chamber 1, and when the first projections are arranged on both opposite side walls or adjacent side walls of the sub-chamber, the first projections 107 on the opposite side walls or adjacent side walls are arranged alternately in the axial direction of the body chamber 1.

Referring to FIG. 6, in an exemplary embodiment, the sub-chamber 11 is provided with at least one pillar 108 on a bottom wall located at the second surface, and multiple second projections 109 are provided at intervals on a peripheral surface of the pillar 108.

The pillar 108 extends in a direction parallel to the axial direction of the body chamber 1.

The shape of the second projections 109 may be a dot shape, a column shape, or a spiral shape, which is not limited herein, and the arrangement of the pillar 108 and the second projection 109 may enhance the mixing effect of various reagents.

Illustratively, at least two columns of second projections 109 are arranged to be spaced apart along a circumferential direction of the pillar 108, and two adjacent columns of second projections 109 are arranged alternately in an extending direction of the pillar 108.

In an exemplary embodiment, multiple third projections are arranged to be spaced apart on an inner wall of the first accommodating chamber 23. The shape of the third projection may be a dot shape, a column shape or a spiral shape, which is not limited herein, and the arrangement of the third projections may enhance the mixing effect of different reagents.

In an exemplary embodiment, the plurality of sub-chambers 11 includes an empty chamber for re-mixing of the sample with different reagents, an inner wall of the empty chamber is provided with fourth projections which are arranged to be spaced apart at intervals.

By providing the empty chamber, a mixed solution obtained in a certain process step can enter the empty chamber for a second mixture, and the mixing effect can be enhanced under a cooperation of the stretch-out and draw-back movement of the piston 3.

In an exemplary embodiment, the piston includes a movable portion 31 and a transmission portion 32 connected to the movable portion 31, the movable portion 31 extends into the second accommodating chamber 24, an orthographic projection of the movable portion 31 in the axial direction of the second accommodating chamber 24 fully covers the second accommodating chamber 24, the transmission portion 32 is located outside the second accommodating chamber, and the transmission portion 32 includes a transmission gear. Under driving of the transmission gear, a synchronous rotation of the piston and the piston rotatable plate can be controlled, and there is no need to provide a separate, additional driving structure for the piston rotatable plate.

In the exemplary embodiment, a sliding groove is arranged on the inner wall of the second accommodating chamber 24 along the axial direction of the second accommodating chamber 24, and a sliding block fitting with the sliding groove is arranged on a peripheral surface of the movable portion 31.

The arrangement of the sliding groove facilitates the stretch-out and draw-back movement of the sliding groove along the axial direction of the second accommodating chamber 24, which prevents a relative movement of the piston 3 in the circumferential direction of the second accommodating chamber 24, and facilitates the rotation of the piston rotatable plate 2 driven by the piston 3.

In an exemplary embodiment, the movable portion 31 is made of an elastic material, and a cross-sectional shape of the movable portion 31 conforms to a shape of the second accommodating chamber 24 in a direction parallel to the third surface.

As the movable portion 31 is made of an elastic material, the sealing between the piston 3 and the inner wall of the second accommodating chamber 24 can be improved to prevent liquid leakage. As the cross-sectional shape of the movable portion 31 conforms to the shape of the second accommodating chamber 24, the sealing between the piston 3 and the inner wall of the second accommodating chamber 24 can be further improved. The cross-sectional shape of the movable portion 31 in the direction parallel to the third surface can be elliptical, hexagonal, triangular, quadrilateral, and the like. By arranging the movable portion 31 in a special shape (not circular shape), the rotation of the piston rotatable plate 2 driven by the piston 3 can be advantageously improved.

Illustratively, when the shape of the cross-section of the movable portion 31 in the direction parallel to the third surface is a polygon, corners of the polygon are rounded angle, to avoid sharp corner angles and thus to reduce liquid and air leakage.

In an exemplary embodiment, the transmission portion 32 includes a transmission gear. The transmission gear is connected to a driving structure, and can perform a rotary motion or the like under the control of the driving structure.

Referring to FIG. 9, in an exemplary embodiment, a face of the cover 4 facing the body chamber 1 includes a plurality of fastening regions 43 corresponding to the plurality of sub-chambers 11, each of the fastening regions 43 including a projection pattern capable of being fastened to a corresponding sub-chamber 11, the projection pattern has a shape conforming to a cross-sectional shape of the corresponding sub-chamber 11 in a direction parallel to the cover 4, so as to realize a sealing of each sub-chamber 11.

It should be noted that after a preset reagent is contained in a corresponding sub-chamber 11 of the body chamber 1, the cover 4 is fixedly connected to the body chamber 1 and cannot be opened by a user. For example, the cover 4 can be welded to the body chamber 1 to avoid a contamination of the reagent.

An embodiment of the present disclosure further provide a nucleic acid extraction method, in which the above-mentioned detection device is used to perform a detection, where a magnet structure 6 is contained in a clamping slot of a piston rotatable plate 2, the first sub-chamber is a sample chamber for containing a magnetic bead buffer solution, and the plurality of sub-chambers further include a first reagent chamber for containing a binding solution, a second reagent chamber for containing a cleaning solution, a third reagent chamber for containing an eluent, a waste liquid chamber, a PCR reagent chamber for containing a PCR reagent and a detection chamber for containing a detection chip 5.

The nucleic acid detection method includes the following steps:
placing a sample into the sample chamber so that the sample is mixed with the magnetic bead buffer solution to obtain a first magnetic bead mixed solution;
rotating the piston 3 thereby driving the piston rotatable plate 2 to rotate synchronously, to cause the third opening 22 of the piston rotatable plate 2 to be opposite to the first opening 101 of the sample chamber, moving the piston 3 in a direction away from the first accommodating chamber 23, to cause the magnetic bead mixed solution to enter the first accommodating chamber 23, and adsorbing magnetic beads by the magnet structure 6;
rotating the piston rotatable plate 2, to cause the third opening 22 to be opposite to the first opening 101 of the waste liquid chamber, and moving the piston 3 in a direction towards the first accommodating chamber 23, to cause waste liquid from which the magnetic beads are separated to enter the waste liquid chamber;
rotating the piston rotatable plate 2, to cause the third opening 22 to be opposite to the first opening 101 of the first reagent chamber, moving the piston 3 in the direction away from the first accommodating chamber 23, to cause the binding solution in the first reagent chamber to enter the first accommodating chamber 23, and releasing the adsorbed magnetic beads by the magnet structure 6, so that the magnetic beads are mixed with the binding solution to obtain a second mixed solution;
rotating the piston rotatable plate 2, to cause the third opening 22 to be opposite to the first opening 101 of the waste liquid chamber, adsorbing magnetic beads by the magnet structure 6, moving the piston 3 in a direction towards the first accommodating chamber 23 to cause waste liquid from which the magnetic beads are separated to enter the waste liquid chamber;
rotating the piston rotatable plate 2, to cause the third opening 22 to be opposite to the first opening 101 of the second reagent chamber, moving the piston 3 in the direction away from the first accommodating chamber 23, to cause the cleaning solution in the second reagent chamber to enter the first accommodating chamber 23, and releasing the adsorbed magnetic beads by the magnet structure 6, so that the magnetic beads are mixed with the cleaning solution to obtain a third mixed solution;
rotating the piston rotatable plate 2, to cause the third opening 22 to be opposite to the first opening 101 of the waste liquid chamber, adsorbing magnetic beads by the magnet structure 6, and moving the piston 3 in the direction towards the first accommodating chamber 23 to cause waste liquid from which the magnetic beads are separated to enter the waste liquid chamber;
rotating the piston rotatable plate 2, to cause the third opening 22 to be opposite to the first opening 101 of the third reagent chamber, moving the piston 3 in the direction away from the first accommodating chamber 23, to cause the eluent in the third reagent chamber to enter the first accommodating chamber 23, releasing the adsorbed magnetic beads by the magnet structure 6, such that the magnetic beads are mixed with the eluent, and nucleic acid is separated from the magnetic beads to obtain a nucleic acid mixed solution;
rotating the piston rotatable plate 2, to cause the third opening 22 to be opposite to the first opening 101 of the PCR reagent chamber, adsorbing magnetic beads by the magnet structure 6, and moving the piston 3 in the direction towards the first accommodating chamber 23, to cause the nucleic acid mixed solution in the first accommodating chamber 23 to enter the PCR reagent chamber to obtain a fourth mixed solution;
moving the piston 3 in the direction away from the first accommodating chamber 23, to cause the fourth mixed solution to enter the first accommodating chamber 23; and
rotating the piston rotatable plate 2, to cause the third opening 22 to be opposite to the first opening 101 of the detection chamber, moving the piston 3 in the direction towards the first accommodating chamber 23 to cause the fourth mixed solution to enter the detection chamber.

In an embodiment, in the step of rotating the piston rotatable plate 2, to cause the third opening 22 to be opposite to the first opening 101 of the first reagent chamber, moving the piston 3 in the direction away from the first accommodating chamber 23, to cause the binding solution in the first reagent chamber to enter the first accommodating chamber 23, and releasing the adsorbed magnetic beads by the magnet structure 6, so that the magnetic beads are mixed with the binding solution to obtain a second mixed solution, the binding solution enhances the binding effect of the nucleic acid and the magnetic beads, and the step includes the following steps:
rotating the piston rotatable plate 2, to cause the third opening 22 to be opposite to the first opening 101 of the first reagent chamber; and moving the piston 3 in the direction away from the first accommodating chamber 23, so that the binding solution in the first reagent chamber enters the first accommodating chamber 23;
releasing the adsorbed magnetic beads by the magnet structure 6, and rotating the piston rotatable plate 2, to cause the third opening 22 to be opposite to the first opening 101 of the first reagent chamber; moving the piston 3 in the direction towards the first accommodating chamber 23, so that the second mixed solution obtained by mixing the magnetic beads with the binding solution enters the first reagent chamber again; and
moving the piston 3 in the direction away from the first accommodating chamber 23 to cause the second mixed solution to enter the first accommodating chamber 23.

By using the above procedure, the binding solution and the magnetic beads can be sufficiently mixed.

In an embodiment, the step of rotating the piston rotatable plate 2, to cause the third opening 22 to be opposite to the first opening 101 of the second reagent chamber, moving the piston 3 in the direction away from the first accommodating chamber 23, to cause the cleaning solution in the second reagent chamber to enter the first accommodating chamber 23, and releasing the adsorbed magnetic beads by the magnet structure 6, so that the magnetic beads are mixed with the cleaning solution to obtain a third mixed solution may specifically include the following steps:
rotating the piston rotatable plate 2 to cause the third opening 22 to be opposite to the first opening 101 of the second reagent chamber; and moving the piston 3 in the direction away from the first accommodating chamber 23, so that the cleaning solution in the second reagent chamber enters the first accommodating chamber 23;
releasing the adsorbed magnetic beads by the magnet structure 6, and rotating the piston rotatable plate 2, so that the third opening 22 is opposite to the first opening 101 of the second reagent chamber; moving the piston 3 in the direction towards the first accommodating chamber 23, so that the third mixed solution obtained by mixing the magnetic beads with the cleaning solution enters the second reagent chamber again; and
moving the piston 3 in the direction away from the first accommodating chamber 23 so that the third mixed solution enters the first accommodating chamber 23.

By using the above-mentioned steps, the cleaning solution and the magnetic beads can be sufficiently mixed, the cleaning effect can be improved, and impurities other than the nucleic acid can be effectively removed.

In one embodiment, the rotating the piston rotatable plate 2, to cause the third opening 22 to be opposite to the first opening 101 of the third reagent chamber, moving the piston 3 in the direction away from the first accommodating chamber 23, to cause the eluent in the third reagent chamber to enter the first accommodating chamber 23, releasing the adsorbed magnetic beads by the magnet structure 6, such that the magnetic beads are mixed with the eluent, and nucleic acid is separated from the magnetic beads to obtain a nucleic acid mixed solution may specifically include the following steps:
rotating the piston rotatable plate 2, to cause the third opening 22 to be opposite to the first opening 101 of the third reagent chamber; moving the piston 3 in the direction away from the first accommodating chamber 23 so that the eluent in the third reagent chamber enters the first accommodating chamber 23;
releasing the adsorbed magnetic beads by the magnet structure 6, and rotating the piston rotatable plate 2, so that the third opening 22 is opposite to the first opening 101 of the third reagent chamber; moving the piston 3 in the direction towards the first accommodating chamber 23, so that a fourth mixed solution obtained by mixing the magnetic beads with the eluent enters the third reagent chamber again; and
moving the piston 3 in the direction away from the first accommodating chamber 23 so that the fourth mixed solution enters the first accommodating chamber 23.

By using the above procedure, the eluent and the magnetic beads can be mixed sufficiently to effectively detach the nucleic acid from the magnetic beads.

This embodiment also provides a nucleic acid detection method, where the body chamber 1 includes a detection region 13 and the plurality of sub-chambers 11 distributed along the circumferential direction of the body chamber 1, where the plurality of the sub-chambers 11 include a PCR reagent chamber, the PCR reagent chamber contains a PCR reagent to be mixed with a product treated with various preset reagents, a side wall of the detection region 13 is provided with a first outlet 105 and a first inlet 106, and the first opening located in the second sub-chamber includes a second inlet 103 and a second outlet 104; the second sub-chamber is provided with a pipeline for connecting the first inlet 106 and the second outlet 104, and a pipeline for connecting the second inlet 103 and the first outlet 105.

A nucleic acid product obtained by the above-mentioned nucleic acid extraction method is mixed with the PCR reagent to obtain a PCR reagent mixed solution (namely, the fourth mixed solution) under the cooperation of the rotation of the piston rotatable plate 2 and the stretch-out and draw-back movement of the piston 3. Then, the piston rotatable plate 2 is rotated, the second sub-chamber is connected to the first outlet 105 through the sixth sub-opening, the seventh sub-opening 203, and the second inlet 103. The first inlet 106 and the second outlet 104 are in connection with the first accommodating chamber 23 through the third opening 22 to form a loop. Under the cooperation of the stretch-out and draw-back movement of the piston 3, the PCR reagent mixed solution is enabled to enter a detection portion having a detection chip 5 in a detection instrument for detection via the pipeline.

It is to be understood that the above-described embodiments are merely exemplary embodiments that have been employed to illustrate the principles of the present disclosure, and the disclosure is not limited thereto. It will be apparent to those skilled in the art that various modifications and variations can be made without departing from the spirit or scope of the disclosure, and these modifications and variations fall within the protection scope of this disclosure.

1. A detection device, comprising: a body chamber, a cover, a piston rotatable plate, and a piston;
wherein the body chamber comprises a first surface and a second surface which are opposite to each other, and the first surface is an open surface; along a circumferential direction of the body chamber, the body chamber comprises a plurality of sub-chambers, and an end of each of the plurality of sub-chambers that is away from the first surface is provided with a first opening;
the cover is provided to cover the first surface of the body chamber, and a second opening is arranged on the cover, the second opening is configured to connect with a first sub-chamber of the plurality of sub-chambers;
the piston rotatable plate comprises a cylindrical structure connected to the second surface of the body chamber; in an axial direction of the cylindrical structure, the piston rotatable plate comprises a first accommodating chamber and a second accommodating chamber, the first accommodating chamber and the second accommodating chamber are connected with each other via a connecting channel, and a third surface of the piston rotatable plate facing to the body chamber comprises at least one third opening in connection with the first accommodating chamber; and
the piston is in slidable connection into the second accommodating chamber in an axial direction of the second accommodating chamber, and the piston is synchronously rotatable with the piston rotatable plate in a circumferential direction of the second accommodating chamber.

2. The detection device according to claim 1, wherein in the circumferential direction of the body chamber, the second surface of the body chamber is provided with at least one concentric annular groove recessed toward an interior of the body chamber, each annular groove is provided with at least one first opening, and each third opening is in slidable connection into a corresponding one annular groove.

3. The detection device according to claim 2, wherein the at least one annular groove comprises a first annular groove and a second annular groove, the first opening comprises a first sub-opening located at the first annular groove and at least one second sub-opening located at the second annular groove, the at least one second sub-opening is arranged to be spaced apart on the second annular groove.

4. The detection device according to claim 3, wherein an orthographic projection of the first sub-opening onto the second annular groove is located between two adjacent second sub-openings.

5. The detection device according to claim 3, wherein the at least one third opening comprises a fourth sub-opening and a fifth sub-opening, the fourth sub-opening is in slidable connection into the first annular groove and the fifth sub-opening is in slidable connection into the second annular groove, a first pipeline is arranged between the fourth sub-opening and the first accommodating chamber, and a second pipeline is arranged between the fifth sub-opening and the first accommodating chamber.

6. The detection device according to claim 5, wherein a first filtering membrane is arranged in the first pipeline, the first filtering membrane is fixed on an inner wall of the first pipeline, and an orthographic projection of the first filtering membrane in an axial direction of the first pipeline fully covers the first pipeline.

7. The detection device according to claim 1, wherein a second filtering membrane is arranged in the connecting channel between the first accommodating chamber and the second accommodating chamber, the second filtering membrane is fixed on an inner wall of the connecting channel, and an orthographic projection of the second filtering membrane in an axial direction of the connecting channel fully covers the connecting channel.

8. The detection device according to claim 5, wherein the at least one annular groove further comprises a third annular groove, the at least one third opening further comprises a sixth sub-opening and a seventh sub-opening in slidable connection into the third annular groove, the sixth sub-opening and the seventh sub-opening are arranged to be spaced apart from each other, and a channel is arranged between the sixth sub-opening and the seventh sub-opening.

9. The detection device according to claim 8, wherein the plurality of sub-chambers comprise a second sub-chamber, a side wall of the second sub-chamber is provided with a first outlet and a first inlet, a first opening located in the second sub-chamber comprises a second inlet and a second outlet, a pipeline for connecting the first inlet and the second outlet and a pipeline for connecting the second inlet and the first outlet are arranged in the second sub-chamber, and the second inlet and the second outlet are arranged on the third annular groove.

10. The detection device according to claim 1, wherein a clamping slot is arranged at a center of the third surface, and a connecting column that fits with the clamping slot is arranged at a center of the second surface.

11. The detection device according to claim 10, wherein the connecting column extends to the first surface, an end of the connecting column away from the second surface is arranged to be flush with the first surface, and the plurality of sub-chambers are arranged around the connecting column; and
the connecting column is a hollow structure, and the cover is provided with a fourth opening in connection with a center of the connecting column.

12. The detection device according to claim 2, wherein the first opening comprises at least two connecting pieces connected against each other, which are formed by cutting at a first position of the annular groove, the third opening is made of an elastic material, and a length of the third opening in a direction perpendicular to the third surface is greater than or equal to a depth of the annular groove.

13. The detection device according to claim 12, wherein the first position of the annular groove is recessed towards the interior of the body chamber to be a first groove, the first opening comprises at least two connecting pieces connected against each other, which are formed by cutting at a bottom portion of the first groove, the third opening is made of an elastic material, the length of the third opening in the direction perpendicular to the third surface is greater than the depth of the annular groove, and the length of the third opening in the direction perpendicular to the third surface is greater than or equal to a depth of the first groove.

14. The detection device according to claim 1, wherein a plurality of first projections are arranged at intervals on at least one inner wall of the sub-chamber.

15. The detection device according to claim 14, wherein at least one pillar is arranged at a bottom wall of the sub-chamber located at the second surface, and a plurality of second projections are provided at intervals on a peripheral surface of the at least one pillar.

16. The detection device according to claim 1, wherein a plurality of third projections are arranged at intervals on an inner wall of the first accommodating chamber.

17. The detection device according to claim 1, wherein the piston comprises a movable portion and a transmission portion connected to the movable portion, the movable portion extends into the second accommodating chamber, an orthographic projection of the movable portion in the axial direction of the second accommodating chamber fully covers the second accommodating chamber, the transmission portion is located outside the second accommodating chamber, and the transmission portion comprises a transmission gear.

18. The detection device according to claim 17, wherein a sliding groove is arranged on the inner wall of the second accommodating chamber along the axial direction of the second accommodating chamber, and a sliding block that fits the sliding groove is arranged on a peripheral surface of the movable portion.

19. The detection device according to claim 17, wherein the movable portion is made of an elastic material, and a cross-sectional shape of the movable portion is consistent with a shape of the second accommodating chamber in a direction parallel to the first surface.

20. A nucleic acid extraction method, wherein a detection is performed by using the detection device according to claim 1, a magnet structure is contained in a clamping slot of the piston rotatable plate, the first sub-chamber is a sample chamber for containing a magnetic bead buffer solution, and the plurality of sub-chambers further comprise: a first reagent chamber for containing a binding solution, a second reagent chamber for containing a cleaning solution, a third reagent chamber for containing an eluent, a waste liquid chamber, a PCR reagent chamber for containing a PCR reagent, and a detection chamber for containing a detection chip;
the method comprises:
placing a sample into the sample chamber, to cause the sample to be mixed with the magnetic bead buffer solution to obtain a first magnetic bead mixed solution;
rotating the piston thereby driving the piston rotatable plate to rotate synchronously, to cause the third opening of the piston rotatable plate to be opposite to a first opening of the sample chamber; moving the piston in a direction away from the first accommodating chamber, to cause the magnetic bead mixed solution to enter the first accommodating chamber; and adsorbing magnetic beads by the magnet structure;
rotating the piston rotatable plate, to cause the third opening to be opposite to a first opening of the waste liquid chamber; and moving the piston in a direction towards the first accommodating chamber, to cause waste liquid from which the magnetic beads is separated to enter the waste liquid chamber;
rotating the piston rotatable plate, to cause the third opening to be opposite to a first opening of the first reagent chamber; moving the piston in the direction away from the first accommodating chamber, to cause the binding solution in the first reagent chamber to enter the first accommodating chamber; and releasing the adsorbed magnetic beads by the magnet structure, so that the magnetic beads are mixed with the binding solution to obtain a second mixed solution;
rotating the piston rotatable plate, to cause the third opening to be opposite to the first opening of the waste liquid chamber; adsorbing the magnetic beads by the magnet structure; and moving the piston in the direction towards the first accommodating chamber, to cause waste liquid from which the magnetic beads is separated to enter the waste liquid chamber;
rotating the piston rotatable plate, to cause the third opening to be opposite to a first opening of the second reagent chamber; moving the piston in the direction away from the first accommodating chamber, to cause the cleaning solution in the second reagent chamber to enter the first accommodating chamber; and releasing the adsorbed magnetic beads by the magnet structure, so that the magnetic beads are mixed with the cleaning solution to obtain a third mixed solution;
rotating the piston rotatable plate, to cause the third opening to be opposite to the first opening of the waste liquid chamber; adsorbing the magnetic beads by the magnet structure; moving the piston in the direction towards the first accommodating chamber, to cause waste liquid from which the magnetic beads is separated to enter the waste liquid chamber;
rotating the piston rotatable plate, to cause the third opening to be opposite to a first opening of the third reagent chamber; moving the piston in the direction away from the first accommodating chamber, to cause the eluent in the third reagent chamber to enter the first accommodating chamber; and releasing the adsorbed magnetic beads by the magnet structure, so that the magnetic beads is mixed with the eluent and nucleic acid are separated from the magnetic beads to obtain a nucleic acid mixed solution;
rotating the piston rotatable plate, to cause the third opening to be opposite to a first opening of the PCR reagent chamber; moving the piston in the direction towards the first accommodating chamber, to cause the nucleic acid mixed solution to enter the PCR reagent chamber, wherein the nucleic acid mixed solution mixes with the PCR reagent in the PCR reagent chamber to obtain a PCR reagent mixed solution;
moving the piston in the direction away from the first accommodating chamber, to cause the PCR reagent mixed solution to enter the first accommodating chamber; and
rotating the piston rotatable plate and moving the piston is the direction towards the first accommodating chamber, to cause the PCR reagent mixed solution to enter a detection region for detection.
